(19) United States
(12) Patent Application Publication
(10) Pub. No.: US 2015/0366438 A1
(43) Pub. Date: Dec. 24, 2015

(54) METHODS AND STEERING DEVICE FOR MINIMALLY INVASIVE VISUALIZATION SURGERY SYSTEMS

(71) Applicant: Vantage Surgical Systems Inc., Irvine, CA (US)

(72) Inventors: Jason Wilson, Los Angeles, CA (US); Vacit Arat, La Canada Flintridge, CA (US)

(73) Assignee: VANTAGE SURGICAL SYSTEMS INC., Irvine, CA (US)

(21) Appl. No.: 14/727,023

(22) Filed: Jun. 1, 2015

Related U.S. Application Data
(63) Continuation-in-part of application No. 14/011,493, filed on Aug. 27, 2013.
Application No. 14/011,493 is a continuation-in-part of application No. 13/761,136, filed on Feb. 6, 2013.
(60) Provisional application No. 61/595,467, filed on Feb. 6, 2012.

(51) Int. Cl. A61B 1/00; A61B 1/04; A61B 17/00

(52) U.S. Cl. CPC A61B 1/00149; A61B 17/00234; A61B 1/04; A61B 1/00128; A61B 1/00039; A61B 1/00066; A61B 1/0002; A61B 1/00027

(57) ABSTRACT

Methods and a steering device for minimally invasive visualization surgery system are disclosed. In particular, the steering device configured for holding and positioning an image capturing device about a frame above a surgical site. The steering device including a curvilinear prismatic joint which may be operated by an end effector and a braking system including one or more sensors. In some embodiments, one or more electrical power mechanisms can be used to inhibit motion according to predetermined parameters can be included. A controller in logical communication with a database including said predetermined parameters (including, for example, ranges of motion for the curvilinear prismatic joint) may also be used to control the range of motion using the one or more electrical power mechanisms and/or the braking system. The braking system can include one or more of a frictional brake, a pumping brake, and an electromagnetic brake.

### RELATED APPLICATIONS

This application claims priority, as a Continuation in Part, to U.S. Non-Provisional patent application Ser. No. 14/011,493 filed Aug. 27, 2013 titled “Stereoscopic System for Minimally Invasive Surgery Visualization” being incorporated herein by reference as if set forth in its entirety.

### FIELD OF THE INVENTION

The present invention relates generally to the field of minimally invasive surgery (MIS) and more particularly to improved methods and devices for use in visualization surgical systems.

### BACKGROUND OF THE INVENTION

Unlike in traditional open surgery procedures, with the use of visualization systems, minimally invasive surgeries (MIS) allow practitioners to perform surgical procedures via small incisions for the same common purpose. These MIS controlled procedures are beneficial to the patient in that they result in less damage to biological tissue and thus less pain, scarring and faster recoveries. More importantly, by reducing the incision size and damage to biological tissue, MIS helps minimize the risk of infections, and in most cases, the overall cost of the surgical treatment.

Passive and active visualization systems are currently being used by practitioners to perform MIS. Both of these types of systems can include fiber optic cables, miniature video cameras and instruments which have been redesigned to be handled via tubes inserted in the small incision(s) to enable the practitioner to perform the procedure while indirectly viewing images/video of the surgical site that are transmitted to external monitors. Because MISs are so different than the traditional open surgery procedures, significant learning and practice is required from practitioners, including for example simulated learning cadaveric training, to be able to operate these systems that enable MIS but require indirect visualization of the surgical site.

MIS visualization systems use hand held endoscopes for capturing the surgical site images displayed on the monitor(s). In using hand held endoscopes however, the practitioner often has to give up visualization control to an assistant (assistant surgeon, attending nurse, etc.) to steer the endoscope via verbal instructions from him/her. With instructions from the practitioner, the assistant rotates the endoscope about the surgical incision to view different locations of the surgical area and/or physically translates the endoscope through the incision closer to the tissue to view magnified views.

In order to free the assistant from the camera steering task or retain control by the practitioner, two types of endoscope positioning arms for the MIS visualization systems have been developed. These arms fall into two categories—passive and active. Passive means that there are no actuators. The user must manually move the endoscope to the correct location and then the arm is locked into place maintaining the endoscope viewing directions. Active means that actuators are attached to the arm articulating joints, allowing it to be teleoperated via some human machine interface (HMI) or in a robotic fashion. For both active and passive types there are two common vital properties in need of much improvement to facilitate learning and operating the use of these systems in the operating room—(1) improved intuitive user interface, and (2) minimizing intrusiveness to the surgeon in the surgical field (i.e. low profile).

With both active and passive systems, when operating an endoscope positioning arm during surgery the surgeon, or assistant, may need to intermittently move the endoscope to a particular location to image the appropriate surgical field tissues. Once in location, the surgeon or assistant may return to other critical tasks while the positioning arm holds the endoscope in place. In the case of the surgeon steering the endoscope, any time spent interacting with the positioning arm is time taken away from surgical task(s) and requires him/her to re-orient himself/herself with the new perspective resulting from the movement and being projected by the monitor, which is outside the line of sight of the surgeon. Currently active arms have implemented different control modalities including joystick, voice, and head mounted trackers, for example. Most commercialized versions of these devices have been discontinued or simply not found in many operating rooms due to their difficulty to use. One exception is the da Vinci surgical system by Intuitive Surgical™. This device uses the joystick paradigm for remote control. Practitioners have learned and continue to implement this device, at least in part due to the joystick which can control both the surgical instruments and visualization, thus allowing the use of a single HMI for all surgical tasks. However, this system is extremely complex (e.g. due to its cost and constant calibration requirements) and in some MIS procedures where it is preferred for the practitioner to manipulate the instruments directly—the system's practicality is contradicted and often results in an obstruction to the practitioner.

Similarly, the available passive positioning arms also suffer from a cumbersome HMIs. In these system, by nature the operator typically must loosen joints separately from the adjustment of the endoscope resulting in a time consuming process in need of much improvement. Further, some additionally suffer from redundant degrees of freedom which while allowing for versatile positioning of the arm and the endoscope, require additional time, from the surgeon or assistant, to adjust the pose of the arm as well as the position of the endoscope at times resulting in a hazard during surgery.

Accordingly, methods and devices for improved MIS visualization systems are desired to overcome the aforementioned problems by providing more intuitive and faster visualization adjustments and operations.

### SUMMARY OF THE INVENTION

The foregoing needs are met, to a great extent, by the present invention, wherein in some aspects of embodiments of the invention are intended to address one or more of the above noted fundamental problems associated with visualization systems used in conventional minimally invasive surgery. More specifically, an improved method and steering device is taught to provide an intuitive MIS visualization system. The improved positioning methods and device of the various embodiments of the invention are applicable to many types of minimally invasive surgery. For example, the method and system may be used in the areas of thoracoscopic, laparoscopic, pelviscopic, arthroscopic, ophthalmics, and other MISs which may be currently approved or in development. For laparoscopic surgery, significant utility can be found of aspects of the present disclose for cholecystectomy, hernia repair, bariatric procedures (bypass, banding, sleeve, or the like), bowel resection, hysterectomy, appendectomy, gastric/anti-reflux procedures, and nephrectomy.

For example, in one aspect of the disclosure, one or more of the problems can be addressed by returning direct control of an imaging device to the surgeon, located in the surgical field, via a novel positioning arm.

According to some aspects of the disclosure, a steering device for holding and positioning an image capturing device of a minimally invasive visualization system is disclosed. The steering device including a first joint assembly mounted to a base, a curvilinear prismatic joint having a proximate and a distal end, wherein the proximate end is connected to the first joint assembly and the distal end is connected to a second joint assembly, an end effector connected to the second joint assembly and configured to secure the imaging capturing device, and a braking system having a user interface configured to lock at least the curvilinear prismatic joint to a fixed position. In some embodiments, one or more electrical power mechanisms used to inhibit motion of the end effector holding the imaging device according to predetermined parameters can be included. A controller in logical communication with a database including said predetermined parameters may be used to control the range of motion using the one or more electrical power mechanisms and/or the braking system. According to some aspects, the predetermined parameters include, for example, ranges of motion for the curvilinear prismatic joint. The braking system can include one or more of a frictional brake, a pumping brake (e.g., hydraulic brake), and an electromagnetic brake.

According to additional aspects of the disclosure, a corresponding method for a steering device holding and positioning an image capturing device of a minimally invasive visualization system is disclosed. The method including determining a frame that is above a surgical area, in a sterile field, and within reach of a surgeon performing a minimally invasive visualization procedure; configuring an end effector located within the frame and connected to a curvilinear prismatic joint to hold an image capturing device; configuring the curvilinear prismatic joint to move the end effector holding the image capturing device within the determined frame; and configuring a range of motion of the curvilinear prismatic joint to be controlled by one or more sensors of the end effector.

According to aspects of the disclosure, the configuration enables the steering device's kinematics to not be redundant and avoid kinematic singularities allowing the surgeon or assistant to manipulate the imaging device without safety concerns for the desired pose.

There has thus been outlined, rather broadly, certain aspects of the invention in order that the detailed description thereof herein may be better understood, and in order that the present contribution to the art may be better appreciated. There are, of course, additional aspects of the invention that will be described below and which will form the subject matter of the claims appended hereto.

In this respect, before explaining at least one aspect of the invention in detail, it is to be understood that the invention is not limited in its application to the details of construction and to the arrangements of the components set forth in the following description or illustrated in the drawings. The invention is capable of aspects in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the invention. It is important, therefore, that the claims be regarded as including such equivalent constructions insofar as they do not depart from the spirit and scope of the invention.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention.

FIG. 1 is a perspective view of a typical general-purpose passive type positioning system being used as an endoscope holder.

FIG. 2 is a diagram of the configuration of a da Vinci® active type visualization system is depicted.

FIG. 2A is a magnified view of a section of the FIG. 2 active type system showing the hands of a practitioner remotely operating the system.

FIG. 3A is a side view of an exemplary imaging device which may be implemented in a MIS visualization system according to aspects of the disclosure.

FIG. 3B is a side view of an exemplary percutaneous cannula device which be implemented in a MIS visualization system according to aspects of the disclosure.

FIG. 3C is a cross section of a patient's skin is shown with the exemplary imaging device of FIG. 2A positioned inside the exemplary percutaneous cannula of FIG. 1B according to aspects of the present disclosure.

FIG. 4 is a perspective view of an exemplary embodiment of the steering device using the surgical table as a base.

FIG. 5 is a perspective view of an exemplary lower gimbal used to attach the steering device of FIG. 4 to the surgical table.

FIG. 6 is a cross-section view of an exemplary braking system including a carriage assembly with a brake assembly.

FIG. 7, is a cross section view of an exemplary curvilinear prismatic joint.

FIG. 8 is a cross section of an exemplary braking system for the curvilinear prismatic joint.

FIG. 9A is a side view of the exemplary steering device being aiming down towards the patient's feet.

FIG. 9B is a side view of the exemplary steering device being aimed towards the center and towards the patient's back.

FIG. 9C is a side view of the exemplary steering device being aimed up towards the patient's head.

FIG. 10A is a bottom view of the steering device with the imaging device aimed towards the right of the patient.

FIG. 10B is a bottom view of the steering device with the imaging capturing device aired towards the left of the patient.

FIG. 11 is a schematic view depicting exemplary components that may be included in some embodiments of the steering device.

FIG. 12 is a flowchart showing exemplary steps that may be used to implement the steering system according to aspects of the present disclosure.

The present invention is further described in the detailed description that follows.

### DETAILED DESCRIPTION OF THE INVENTION

### Currently Available MIS Visualization Systems

As described in the background, in the field of MIS there are generally two types of visualization systems that have been developed to allow practitioners to view the surgical area while surgery is being performed.

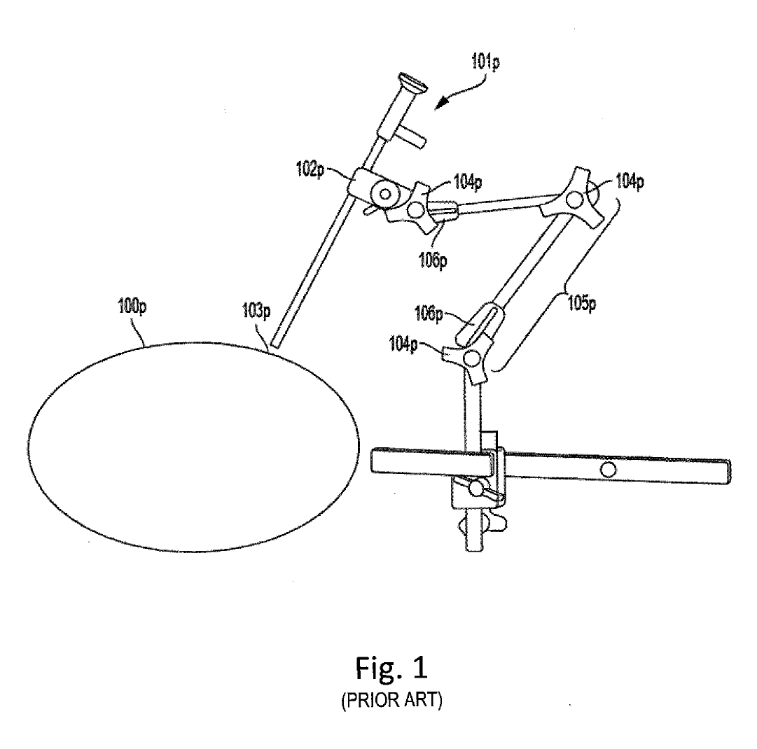

Referring now to FIG. 1, a perspective view of an exemplary general purpose passive type positioning arm that is used as an endoscope holder is shown. The cross section of the patient 100p is represented by an oval with an endoscope 101p passing into the patient 100p. The end effector 102p of the endoscope holder grasps the endoscope at a location above the patient entry point 103p. In order to reposition the endoscope 101p, the surgeon or assistant must loosen one or more of the locking screw(s) 104p, move the endoscope 101p to the desired location, and then tighten the locking screw(s) 104p. This type of positioning arm requires two hands and is a cumbersome and slow procedure. Furthermore, the elbow 105p of the positioning arm must also be separately positioned due to the over-determined kinematics of the arm which is caused by the extra degrees of freedom of the ball joints 106p typically used in these types of generic positioning devices. The extra degrees of freedom are required however precisely because of the limitations of using an elbow 105p. Aside from being time consuming and cumbersome, these limitations decrease the safety for use during MIS resulting in very minimal use by practitioners.

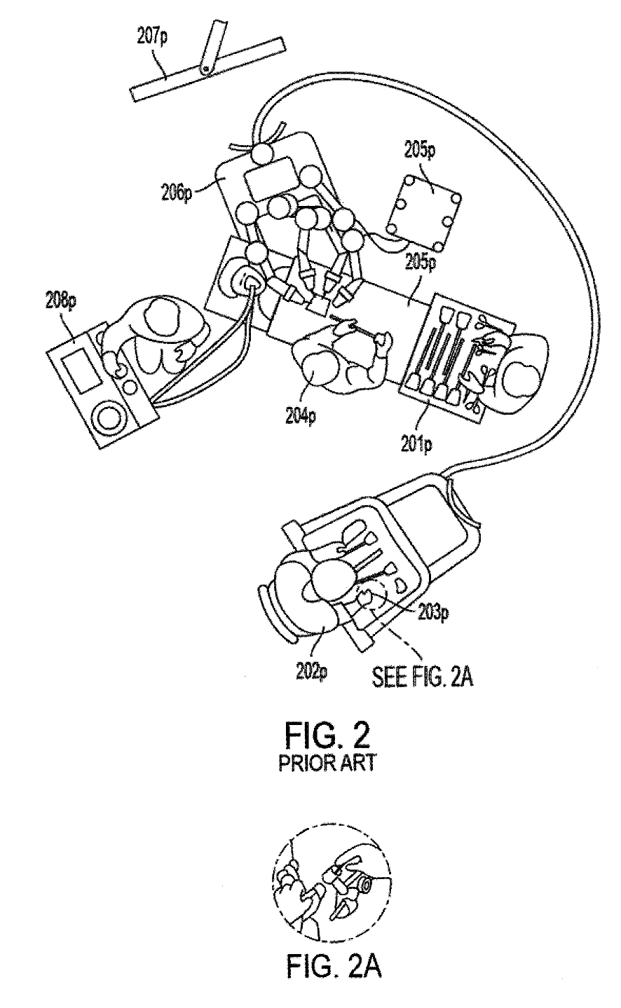

Referring now to FIG. 2, a diagram showing the configuration of an exemplary active type visualization system is depicted. In particular, showing that while the active systems are highly sophisticated, these systems—as previously explained in the background section—all require physicians to undergo significant training so that he/she can step away from the sterile area and perform the surgery remotely. This also requires constant calibration and extra personnel to help perform the surgery adding even more to the costs.

For a typical MIS procedure, a patient can undergo surgery by laying on a surgical bed. Above the surgical area, there is the sterile area 205p and nearby a first cart 206p of the active system is positioned. This first cart 206p is controlled remotely by a surgeon 202p sitting outside of the surgical field 205p (sometimes performing telesurgery from outside the room) sending signals via an operative console 203p. In FIG. 2A, the hands of a practitioner are shown operating the active surgical system in a magnified view of a section of the operative console 203p. With this operative console 203p the surgeon 202p uses surgery like hand movements to control, via the cart's surgical tools, the surgery (as shown in FIG. 2A). An assistant 204p is needed to control the surgical views and verbally communicate to the surgeon 202p the state of the patient from observed visual cues. In order for them to view the surgery the assistant 204p and the nurse 201p need to divert their attention from the patient to a monitor 207p that is located on a wall of the room and also outside of the surgical field 204p. The anesthesiologist is also typically around the patient with his/her own devices 208p. Several things occur when implementing this active system configuration. For example, the surgeon must give up control of the visualization device to the assistant increasing the chances of error, the surgeon must also undergo simulation training in order to operate remotely, the surgeon does not get to experience visual cues of the patient that are often observed outside of the surgical site, the systems robotics require expensive and constant calibration, and the amount of people required and the equipment's large size and redundancy can prevent a surgeon 202p from easily getting to the patient in case of an emergency. As a result of some of the aforementioned, improved MIS systems that can overcome some of these limitations safely and while reducing costs are highly desired.

Going forward, various aspects of the steering device of the present disclose may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present.

Relative terms such as “lower” or “bottom” and “upper” or “top” may be used herein to describe one element's relationship to another element illustrated in the drawings. It will be understood that relative terms are intended to encompass different orientations of the steering device in addition to the orientation depicted in the drawings. By way of example, if aspects of the steering device shown in the drawings are turned over, elements described as being on the “bottom” side of the other elements would then be oriented on the “top” side of the other elements. The term “bottom” can therefore encompass both an orientation of “bottom” and “top” depending on the particular orientation of the apparatus.

Various aspects of the steering device may be illustrated with reference to one or more exemplary embodiments. As used herein, the term “exemplary” means “serving as an example, instance, or illustration,” and should not necessarily be construed as preferred or advantageous over other embodiments of a steering arm disclosed herein.

### GLOSSARY

In this description and claims directed to the disclosure, various terms may be used for which the following definitions will apply:

“Articulated motion”, as used herein, can refer to the different parts of the steering device that allow rotation and/or translational motion up to a pre-configured degree of freedom via the end-effector. For example, the motion provided by each of the joint assemblies and the curvilinear prismatic joint in a frame about the surgical site.

“Home position”, as used herein, can refer to a known and fixed location on the basic coordinate axis of the image capturing device manipulator where it comes to rest, or to an indicated zero position for each axis. In some embodiments, a unique position may be provided for each of various modes/settings that the steering device can be set to.

“Frame”, as used herein, can refer to a coordinate system in the sterile field used to determine a position and orientation of the image capturing manipulator in space, as well as the steering device's position within its model and in relation to the patient and/or the surgical area.

“Joint interpolated motion”, as used herein, can refer to the coordination of the movement of the joints, such that all joints arrive at the desired location simultaneously. In some embodiments, predictable paths that do not interfere with the line of sight of the surgeon and/or the surgical tools during surgery.

“Curvilinear prismatic joint”, as used herein, refers to a circular or generally arched joint that can provide linear sliding movement between two connected bodies. In particular, the curvilinear prismatic joint shape is designed to provide linear movement that by its design contours around a patient's body that is laying on a surgical bed. According to some aspects of the disclosure, the curvilinear prismatic joint can include a braking system that controls the linear movement of the two connected bodies.

“End effector”, as used herein, refers to a tool specifically designed to enable the steering device to perform the intended task of positioning an image capturing device during MIS.

“Sensor”, as used herein, can refer to one or more input devices used to enable a change in position or the fixing of a position of the manipulator relative to the surgical site by sending a resulting signal or data to at least one or more actuator and/or controller. The sensor(s) used may include sensors that respond to physical stimuli (such as heat, light, sound, pressure, magnetism, motion).

“Manipulator”, as used herein, can is a component of the steering arm which is configured to hold the image capturing device and, via the series of rotating and sliding joints, move the position of the image capturing device relative to the surgical site. The control of the manipulator may be by an operator via the end-effector and/or a programmable controller or any logic system (e.g., wired system).

“Imaging device” or “image capturing device”, as used herein, refers to a percutaneous optical visualization device or system, including for example, a percutaneous optical channel device, an endoscope, etc.

The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as one skilled in the art would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure, which is defined solely by the appended claims and applicable law. Moreover, it is noted that like reference numerals represent similar parts throughout the several views of the drawings.

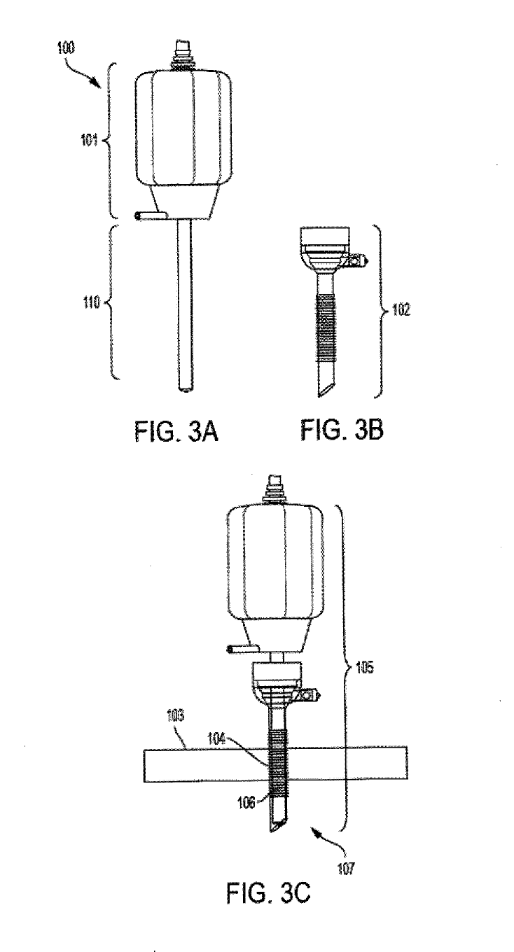

Referring now to FIG. 3A, a side view of an exemplary imaging device is shown. The imaging device 100 may be, for example, an endoscope which can include a tube 110 ranging from 1 mm-20 mm that traverses the patient's skin through an incision to image a surgical site. In this exemplary embodiment, the optics inside can include a distal lens stack followed by a series of relay lenses to bring light from the surgical site to the image capturing device 101 attached proximally as shown. The imaging device 100 can include other optical components and at least one camera to digitally capture images and send them to one or more electronic displays, with at least one preferably being in the line of sight of the surgeon and in the sterile field, allowing the practitioner to perform MIS standing next to the patient as he/she would in open surgery.

The optical channel 110 can pass through a cannula 102, which holds the incision open and can allow free movement of the optical channel 110 portion to be introduced and removed from the surgical site. The cannula 102 may include a plurality of valves and seals to can allow surgical devices to be introduced and removed whilst avoiding the loss of insufflation gases from the internal surgical area. In some embodiments, the cannula 102 may be in logical communication with a user interface (not shown) of some steering device embodiments, to also lock the imaging device 100 prevent it from translating inside/outside of the body.

Referring now to FIG. 3B, a side view of an exemplary percutaneous cannula device 102 which be implemented in a MIS visualization system. In FIG. 3C, the cannula 102 is shown with the optical channel 110 of the imaging device 100 inserted through the cannula 102 to gain access to the surgical site 107 below the skin 103. The imaging device 100, optical channel 101 and cannula 102 as shown form an endoscopic imaging device 105. In some embodiments, the cannula can include a series of ribs 106 or other protruding external features that assist fixing the endoscopic imaging device in the incision 104.

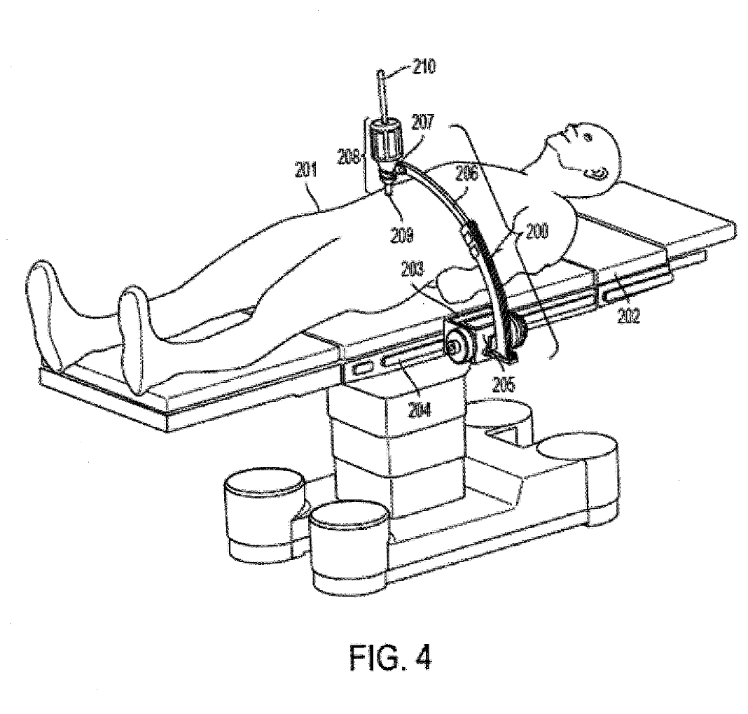

Referring now to FIG. 4, a perspective view of an exemplary embodiment of the steering device with positioning arm 200 supported by a surgical table 201 and used to position the imaging device 105 above a patient 202 lying on the surgical table 201 is shown. In particular, the positioning arm 299 which can include a base 203 which can be attached to a bedrail 204 of a surgical table 201. Attached to the base 203 can be a two degree of freedom gimbal 205. As shown starting from the base 203, the first rotational joint of the gimbal 205 can be parallel to bed rail 204. The second rotational axis can be orthogonal to the first and attached in serial. Attached to the gimbal can be curvilinear prismatic joint, e.g. a circular prismatic joint 206. These three joints in concert can allow positioning of the imaging device in Cartesian coordinates.

Further, the curvilinear prismatic joint can allow for large radius rotational motion about a point in a frame above the patient 201 and the surgical table 202. This point is adjustable depending on the pose of the gimbal. Using this configuration various safety and practical advantages are provided over conventional rotational joints that do not restrict the degrees on freedom and are not compatible with the surgical configuration. Moreover, the use of a linear prismatic joint would not be suitable as it would interfere with the patient and/or the surgical table.

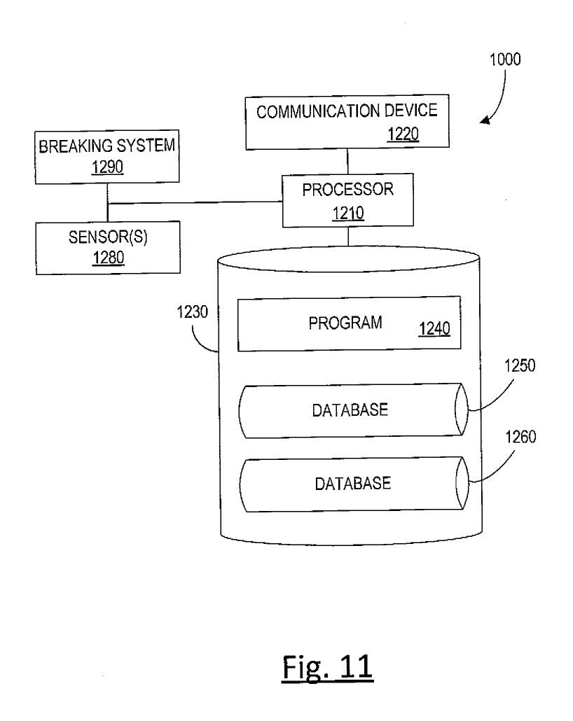

During MIS, it can be desired that the surgeon or assistant be able to rotate the imaging device 208 about a surgical incision 209 to achieve the correct pose of the imaging device 208. This is necessary to ensure imaging of the, and/or accessibility, to specific locations of the surgical site. This can be achieved in part by adding an additional two degrees of freedom via the two rotational joints 207 at the end effector of the steering device. The requirement of these two degrees of freedom is that its constituent rotational joints 207 are oriented such that they do not become parallel to the optical axis of the imaging device 100 during surgery. Ideally the two joints stay as close to orthogonal as possible. The reason for this is the imaging device 100 is free to rotate in the cannula, resulting in a kinematic singularity if a parallel condition occurs. According to some aspects, the angle of rotation of the joints will be limited to less than 90 degrees from a nominal position where the imaging device can be oriented perpendicular to the surgical table (from a table top perspective). The angle of rotation can be limited, for example, by retaining structure that is fixed or by a part capable of being actuated by a controller (shown in FIG. 11) configured to control the range of motion of the manipulator.

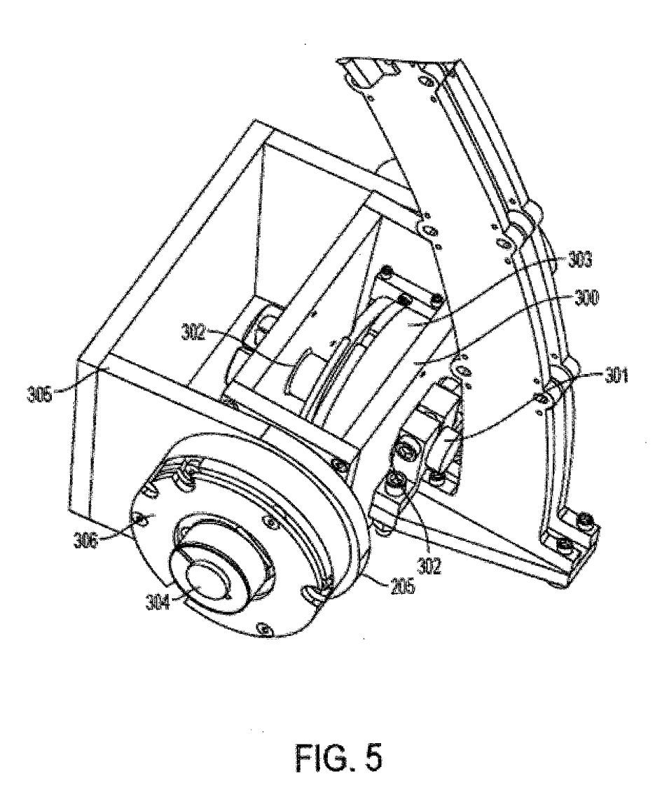

During use, the end effector 210 of the described positioning arm 200 can be attached to some location away from the incision. This could be higher on the length of the imaging device 100, to an image recording device 101, or a coupler attached to the posterior end of the imaging device, for example. In some embodiments, the imaging device 100 and/or image recording device 101 may be ergonomically configured and include sensors to act as the end effector 210, thus eliminating the need for this additional part. The surgeon or assistant may use the end effector 210 to pivot the imaging device 100 about the incision until the desired pose is achieved. Once the desired pose is achieved, the gimbal 205 and circular prismatic joint 206 can lock via a braking system (shown in FIGS. 5, 6 and 8, for example).

If the cannula 102 is sufficiently constrained by the incision 104, the imaging device 100 will be fully constrained and considered “held”. However, if the cannula 102 is not sufficiently constrained, the end effector joints 207 must also lock to fully constrain the imaging device 100 and consider it held. In practice the cannula 102 may be sufficiently constrained by friction between the incision 104 and the cannula 102 external features 106 and thus locking of 207 can be unnecessary.

The utility of this configuration may be that when unlocked, the surgeon or assistant can be free to move the endoscopic imaging device 105, for example, by simply rotating and translating it much as is done without the use of a positioning arm. However, when let go, the locked arm can hold the endoscopic imaging device 105 in place. Due to the unique joint configuration, there is no need to be concerned with the pose of the positioning arm 200 interfering with the procedure, the imaging device falling, calibration and the such.

The surgeon or assistant typically manipulates the endoscopic imaging device by gasping the manipulator 210, which may be part of the imaging device 100/imaging recording device 101, with a hand. In some embodiments, the sensors to lock and unlock the arm may be part of the manipulator 210. The use of capacitive sensors would make the sensors invisible to the surgeon or assistant, making the use of the imaging device steering device almost—if not completely—innocuous to the surgeon or assistant. Capacitive sensors can include any sensor(s) used to detect and measure proximity, position or displacement, humidity, fluid level, and acceleration, for example, known in mouse track pads, touch displays, automotive door handles, industrial fluid indicators, etc. Other/additional sensors that may be desired in some embodiments can include sensor(s) that respond to physical stimuli (such as heat, light, sound, pressure, magnetism, motion) or a signal from a patient monitoring device. A signal from a patient monitoring device may include, for example, a signal relating to the patient's heart rate received by the controller.

In yet additional embodiments, additional safety sensors may be positioned in joints 207, 203, 205, for example, to limit or provide a warning when the range of motion approaches an unsafe position. In some embodiments, the two capacitive sensors may be located on opposite sides of the manipulator 210 to eliminate inadvertent unlocking of the arm due to bumping. Logic circuitry ensure the arm only unlocks when multiple sensors on the manipulator 210 are activated ensures that the arm is unlocking due to a grab event versus a bump event. In additional embodiments, a small vibration device (motor) and/or light may be included in the manipulator 210 to provide a warning to the user about a condition. A condition may include, for example, a heart rate electrical signal falling outside a predetermined threshold, an electrical signal received from the imaging device's sensor located on the distal end sensing the distance to a delicate boundary (e.g. organ), etc.

Referring now to FIG. 5, a perspective view of an exemplary lower gimbal used to attach the steering device of FIG. 4 to the surgical table is depicted. In particular, the second rotational joint of the gimbal including a carriage 300 and an axle 301 which can be configured to be free to rotate relative to the carriage 300 on bearings 302 attached to the carriage 300. As shown, the circular prismatic joint 206 may be fixed to the axle of the gimbals second rotational joint. Attached to the center of the carriage 300 is a brake 303. When the brake is disengaged the axle 301 is free to rotate relative to the carriage. When the brake is engaged the axle 301 and the carriage 300 can become a rigid body.

Fixed to the carriage can be a set of two collinear axles 304 oriented orthogonally to the first axle 301. Each ride on the bearing of a yoke 305 attached to the base 203. This first gimbal joint allows the entire carriage assembly to rotate orthogonally to the second gimbal joint. In some embodiments, a second set of brakes 306 can be attached to the yoke 305 and the axles 304. When the brake is disengaged the axles 304 and thus the carriage assembly and circular prismatic joint 206 are free to rotate relative to the yoke 305. When the brake is engaged the axles 304 and the carriage assembly can become a rigid body.

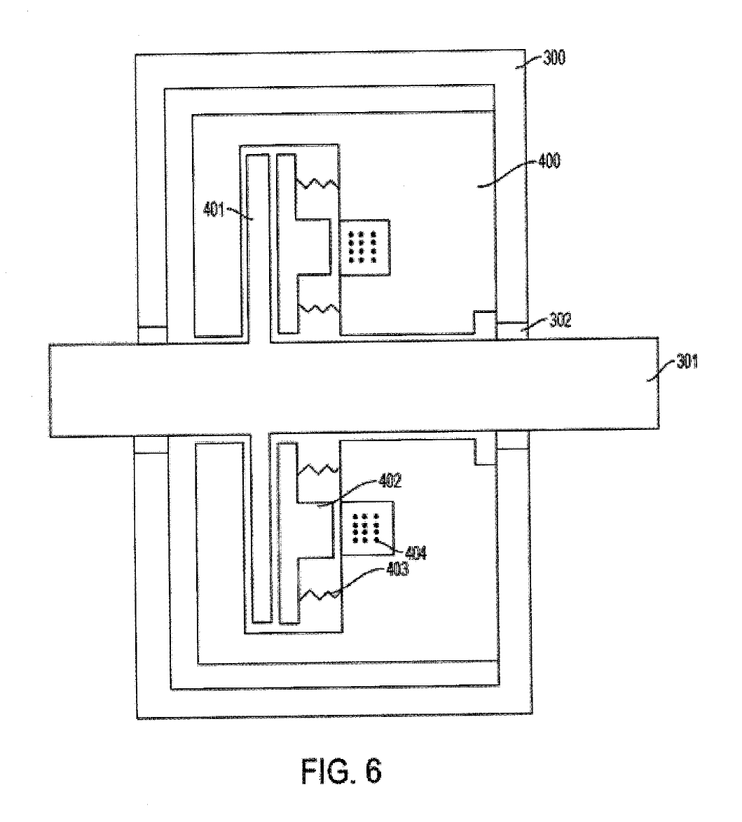

Referring now to FIG. 6, a cross-section view of an exemplary braking system including a carriage assembly with a brake assembly is depicted. As shown in FIG. 5, the axle 301 can be configured to ride on bearings 302 of the carriage 300. Attached to the carriage 300 may be the brake housing 400. Attached to the axle may be a brake disc 401 so that when the brake is engaged or locked, the brake disc 401 can be configured to be held against the brake housing 400 by a pressure plate 402, for example. The pressure plate 402 could then sandwich the brake disc 401 between itself and the brake housing 400 with enough force so that the brake disc 401 and brake housing 400—and thus the carriage 300—remain in a rigid body form as it may be desired to manipulate the steering device.

In one embodiment, the “passive” state of the brake may be locked. In this configuration, a spring 403 can holds the pressure plate 402 against the brake disc 401. To disengage or “unlock” the brake, an actuator must overcome the spring force. Here, an electromagnet 404 may be implemented, for example, and thus is positioned in the vicinity of the pressure plate 403. When current is sent through the electromagnet 404, an attractive force can enact on the pressure plate 403 opposing the spring 403 force disengaging the brake.

In another embodiment, the passive state of the brake may be unlocked. In this configuration, the role of the spring 403 and electromagnet 404 can be reversed. The spring 403 can hold the pressure plate 402 away from the brake disc 401 in the unlocked position. The electromagnet 404 may be placed on the opposite side of the brake housing 400. When current is sent through the electromagnet 404, the pressure plate 402 is attracted to the brake disc 402 and the brake can lock.

As it will be apparent to one skilled in the art, the actuation method is not limited to the electromagnetic type. Alternatively, for example, it may be one or a combination of one or more of a mechanical, pneumatic, hydraulic, electromagnetic and mechatronic system.

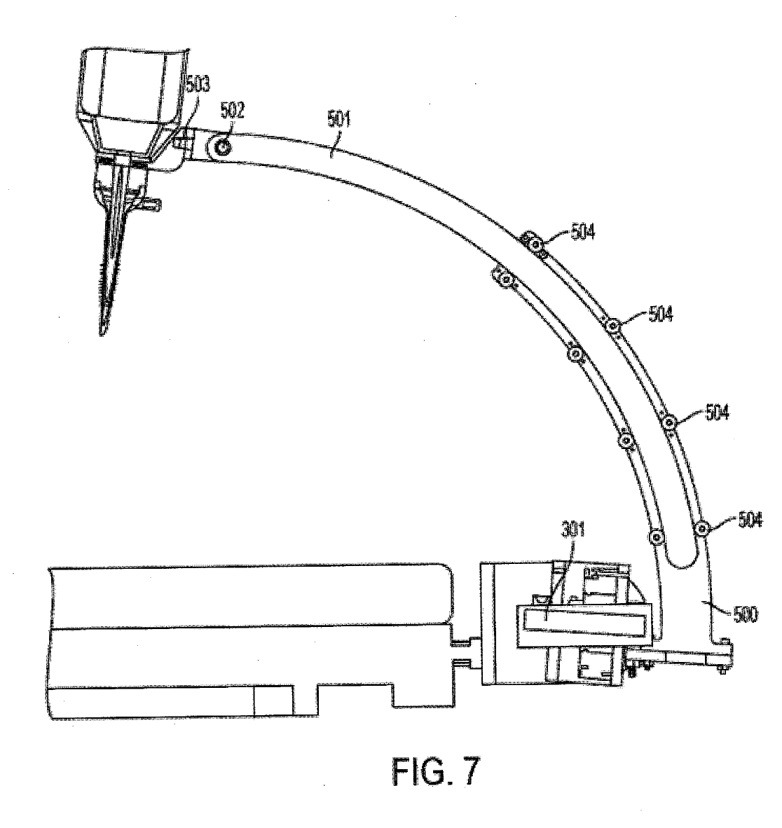

Referring now to FIG. 7, a cross section view of an exemplary curvilinear prismatic joint 200 is shown. In particular, the curvilinear prismatic joint 200 may consists of two distinct components—an exterior slider 500 (analogous to a stator) and an interior slider 501 (analogous to the rotor). As shown, the exterior slider 500 may be attached to the lower gimbal axle 301, and the interior slider 501 may be attached to the upper gimbal 207. The two joints 502 and 503 of the upper gimbal 207 are shown. The first joint 502 can be parallel to the circular motion of the circular prismatic joint 200 while the second joint 503 is orthogonal to the first joint 502 and the optical axis of the imaging device 208. In some embodiments, this configuration may be interchanged while preserving correct operation. This can also be true of the lower gimbal.

The interior slider 501 and exterior slider 500 should have the same radius in at least a portion along the length to allow for proper concentric prismatic motion. In some embodiments, it may be preferred that the entire length of the interior slider 501 and the exterior slider 500 have an arc configuration for a greater range of motion. The interface between the two components must have a low coefficient of friction, either by material choice or a rolling bearing interface to allow for easy manipulation by the surgeon or assistant. In the embodiment shown, for example, the side surfaces of joints are friction slider interfaces, while the radial surfaces interface using a plurality of rollers 504.

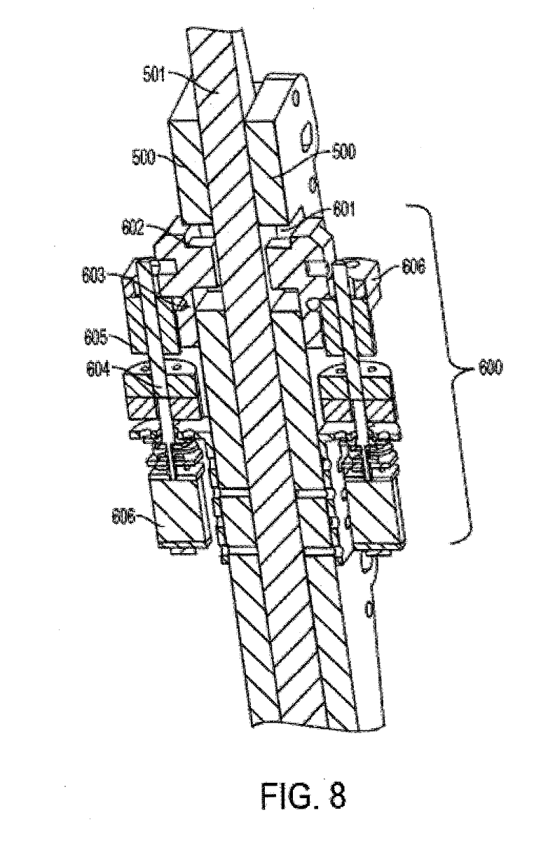

Referring now to FIG. 8, a cross section of an exemplary braking system for the curvilinear prismatic joint 200 is shown. In particular, the interior slider 501 is shown sandwiched between the two walls of the exterior slider 500. Attached to the sides of the exterior slider 500 may be two braking assemblies 600, for example. Holes 601 in the exterior slider 500 can allow brake pads 602 to squeeze the interior slider 501, engaging the brake 600 and “locking” the circular prismatic joint 200. As shown, the brake pads 602 may be attached to the exterior slider 500 to allow rotation about a flexure 603.

The brake pads 602 can be moved towards and away from the interior slider 501 by a lead screw 604. The lead screw 604 rotates and a lead nut 605 can be configured to push against a lever arm of the brake pad 602 rotating it around the flexure 603 and squeezing the inner slider 501. In some embodiments, the lead screws 604 can be rotated by an electric motor. This motor may be one or more of a dc motor, ac motor, servo motor, stepper motor and the like.

The brake pads need not be actuated by a lead screw/motor combination. They can also be actuated with or without a lead screw using solenoids, linear actuators, pneumatic actuators, hydraulic actuators and the like.

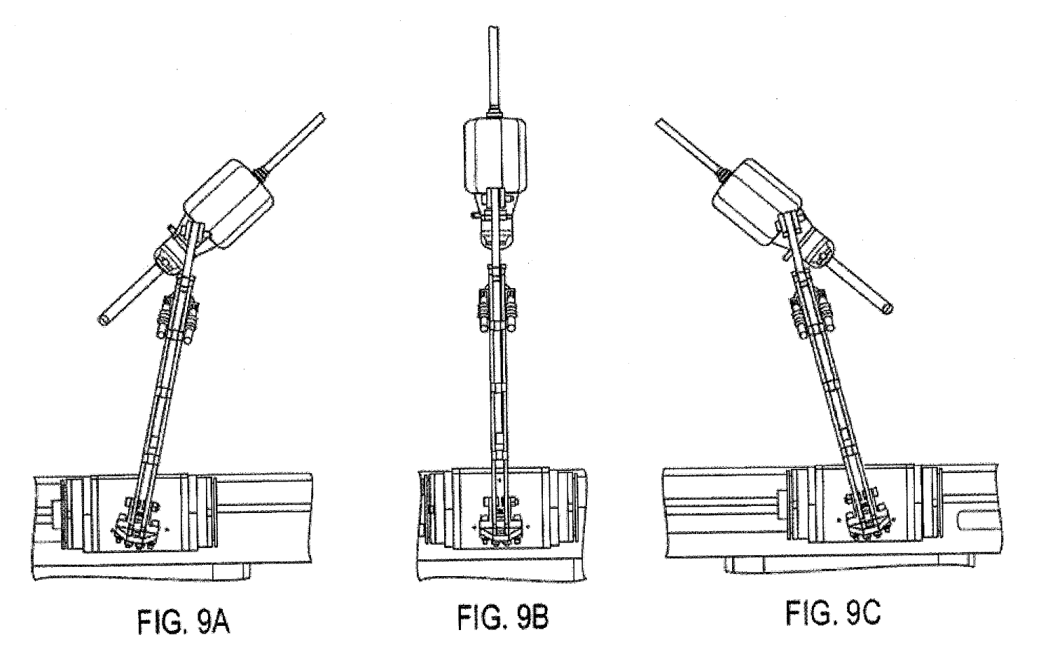

Referring now to FIG. 9A, FIG. 9B, and FIG. 9C, a side view of the positioning arm is shown where the imaging system is rotated around a fixed incision point in which the cannula may be inserted. FIG. 9A shows the endoscopic imaging system pointing “down” towards the location of the patient's feet. FIG. 9B shows the endoscopic centered pointing at the patient's back as shown in FIG. 4. FIG. 7C shows the endoscopic imaging system “up” towards the patient's head. According to some aspects, FIGS. 9A-9C show how the circular prismatic joint and the gimbals can be configured to move in concert to produce the desired joint interpolated motion to tilt about the incision and provide the user the ability to look up and down in the surgical field.

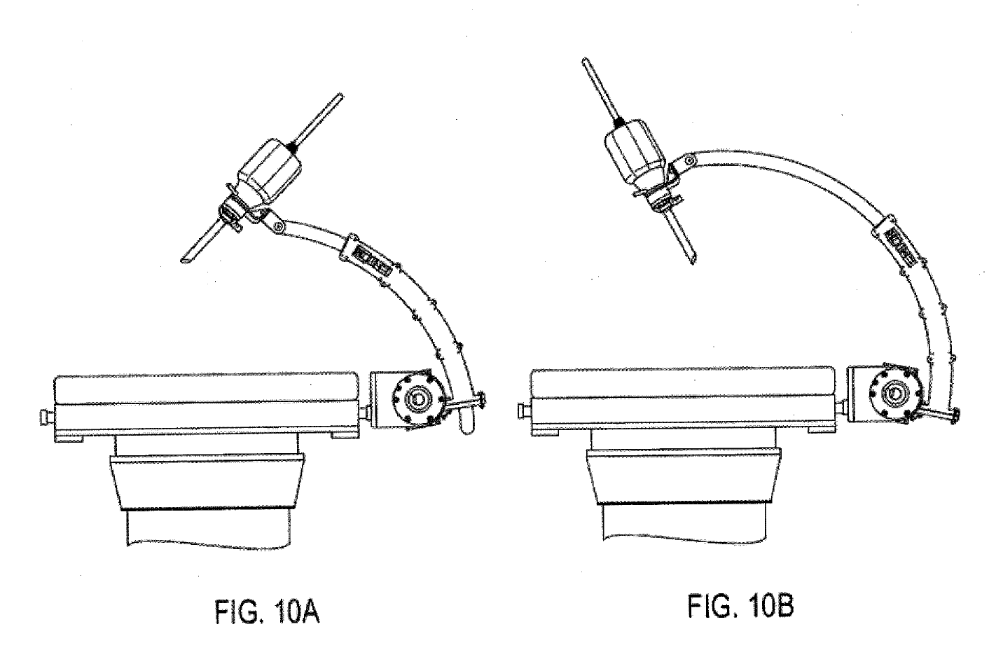

Referring now to FIG. 10A is a side bottom view of the steering device with the imaging device aimed towards the right of the patient. In particular, showing the endoscopic imaging system pointing “right” towards the location of the patient's right side. Referring now to FIG. 10B, the endoscopic imaging system pointing “left” towards the location of the patient's left side. According to some aspects, this figure sequence shows how the circular prismatic joint and the gimbals can provide joint interpolated motion enabling them to move in concert and produce the desired tilting motion about the incision to look left and right in the surgical field.

Referring now to FIG. 11, a schematic view depicting exemplary components that may be included in some embodiments of the steering device are shown. The controller 1000 forming part of the steering device (shown in FIG. 3) can include one or more processors 1210, which may include one or more processor components coupled to a communication device 1220.

The processors 1210 can be coupled to a communication device configured to communicate via a communication channel. The communication device may be used to electronically communicate with networks and/or individual devices, for example, the steering device's interface, a heart monitor, a surgical instrument sensor and the such. The communication device 1220 may also be used to communicate, for example, with one or more additional controller apparatus or programming/interface device components that may provide calibration or manufacturer updates.

The processor 1210 is also in communication with a storage device 230. The storage device 230 may comprise any appropriate information storage device, including combinations of magnetic storage devices, optical storage devices, and/or semiconductor memory devices such as Random Access Memory (RAM) devices and Read Only Memory (ROM) devices.

The storage device 1230 can store a program 1240 for controlling the processor 210. The processor 1210 performs instructions of a software program 1240, and thereby operates in accordance with the present invention. For example, the processor 1210 may lock and/or unlock different braking systems 1290 according to data received from the one or more sensors 1280. The storage device 1230 can also store other pre-determined safety factors, such as rotation parameters and predetermined paths of motion, in one or more databases 1250 and 1260. Accordingly, the database may include, for example, communication protocols, parameters and thresholds, keyword settings, pattern recognition settings, and controlling algorithms for the control of information as well as data and/or feedback that can result from their action. In some embodiments, that data may be ultimately communicated to/from an external device.

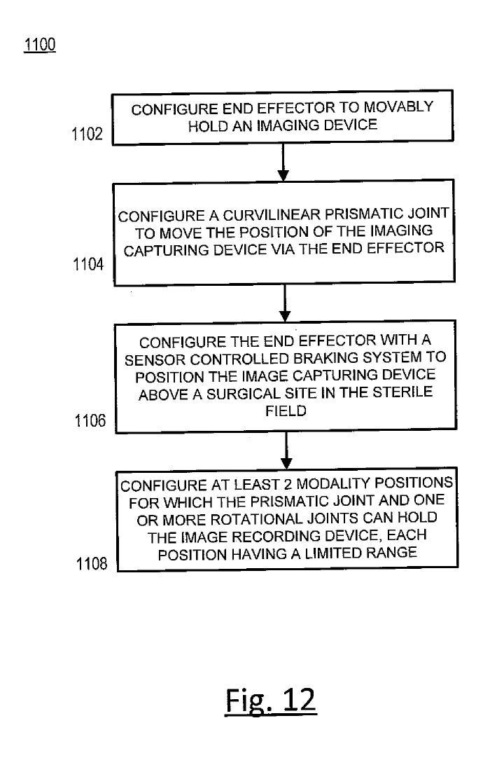

Referring now to FIG. 12, a flowchart with exemplary method steps to implement embodiments according to aspects of the present invention is shown. Beginning at 1102, the end effector can be configured to movably hold an imaging device. At 1104, a curvilinear prismatic joint can be configured to hold the end effector and imaging device above a patient laying on a surgical table. At 1106, a braking system is configured to hold in position the imaging device in relation to a surgical site. In configuring the braking system, in some embodiments at 1108, one or more frames can be configured according to a plurality of modalities. For example, this means that different joints will be engaged/disengaged to limit the movement so that the steering device's movement is controlled all the way to a desired pose. For example, during an emergency, the steering arm is able to provide a path for the steering device to move away from the patient in a manner in which the impact of the removal of the imaging device from the incision and/or cannula is minimized. Other modalities can include, a home operating mode, a cleaning mode, a mode that allows only for controlled precise movement above the surgical site, and the such. Different modalities may be activated by a surgeons input, a received signal from a sensor or a device in communication with the controller, and/or a sensed condition.

The many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to, falling within the scope of the invention.

We claim:

1. A steering device for holding and positioning an image capturing device of a minimally invasive visualization system, the steering device comprising:
a first joint assembly mounted to a base;
a curvilinear prismatic joint having a proximate and a distal end, wherein the proximate end is connected to the first joint assembly and the distal end is connected to a second joint assembly;
an end effector connected to the second joint assembly and configured to secure the imaging capturing device; and
a braking system having a user interface configured to lock at least the curvilinear prismatic joint to a fixed position.

2. The steering device of claim 1, wherein the braking system is additionally configured to lock one or both of the first joint assembly and the second joint assembly, and the first joint assembly and the second joint assembly are rotary joints, each joint assembly being configured to swing a maximum of about 180 degrees about each joint assembly's axis when the braking system is disengaged.

3. The steering device of claim 1 wherein the braking system is additionally configured to lock one or both the first joint assembly and the second joint assembly via a sensor of the user interface.

4. The steering device of claim 3 wherein the locking of the curvilinear prismatic joint and one or both the first joint assembly and the second joint assembly by the braking system, and via the user interface, is synchronized to disengage when the sensor is actuated.

5. The steering device of claim 4 wherein the sensor is mounted on a handle forming part of the end effector and can be used to sense when a user grasps the handle to manipulate the position of the image capturing device.

6. The steering device of claim 1 wherein the curvilinear prismatic joint comprises:
an internal circular part having a distal end and a proximal end, the proximal end being slidably fixed to an end of a complementary external circular part; and
wherein the brake is configured to lock, via a sensor's signal, the internal circular part and the complementary external circular part to either a limited range of motion that is less than two feet or a secure fixed position, according to the sensor's signal.

7. The steering device of claim 1, additionally comprising:
one or more electrical power mechanisms used to inhibit motion of the end effector holding the imaging device according to predetermined parameters; and
a controller in logical communication with a database and the one or more electrical power mechanisms, the database used to store said predetermined parameters used to control the range of motion of the one or more electrical power mechanisms,
wherein the predetermined parameters include a range of motion of the curvilinear prismatic joint.

8. The steering device of claim 1, wherein the braking system includes one or more of a frictional brake, a pumping brake, and an electromagnetic brake.

9. A steering device for holding and positioning an image capturing device of a minimally invasive visualization system, the steering device comprising:
a first rotary joint assembly mounted to a base;
a curvilinear prismatic joint having a proximate and a distal end, wherein the proximate end is connected to the first rotary joint assembly and the distal end is connected to a second rotary joint assembly; and
an end effector connected to the second rotary joint assembly and configured to secure the imaging capturing device.

10. The steering device of claim 9, additionally comprising:
a braking system having a user interface configured to lock at least the curvilinear prismatic joint to a fixed position.

11. The steering device of claim 10, wherein the braking system is additionally configured to lock one or both of the first rotary joint assembly and the second rotary joint assembly via a sensor of the user interface.

12. The steering device of claim 11, additionally comprising:
a controller in communication with the sensor and configured to control at least one actuator of the braking system.

13. The steering device of claim 12, additionally comprising:
a communication device in communication with the controller, the communication device receiving a signal from at least one additional sensor not located on the steering arm.

14. The steering device of claim 12, wherein the braking system includes one or more of a frictional brake, a pumping brake, and an electromagnetic brake.

15. The steering device of claim 12, additionally comprising:
one or more electrical power mechanisms used to inhibit motion of the end effector holding the imaging device according to predetermined parameters stored in a database in communication with the controller,
wherein the predetermined parameters include a range of motion of the curvilinear prismatic joint.

16. A method for a steering device holding and positioning an image capturing device of a minimally invasive visualization system, the method comprising:
determining a frame that is above a surgical area, in a sterile field, and within reach of a surgeon performing a minimally invasive visualization procedure;
configuring an end effector located within the frame and connected to a curvilinear prismatic joint to hold an image capturing device;
configuring the curvilinear prismatic joint to move the end effector holding the image capturing device within the determined frame; and
configuring a range of motion of the curvilinear prismatic joint to be controlled by one or more sensors.

17. The method of claim 16, wherein at least the one or more sensors include a capacitance sensor located on the end effector to enable actuation of the braking system by grasping.

18. The method of claim 16, additionally comprising:
determining at least one additional frame according to predetermined parameters of a modality.
